(19) United States
(12) Patent Application Publication
(10) Pub. No.: US 2026/0021263 A1
(43) Pub. Date: Jan. 22, 2026

(54) NOISE-REDUCING AIR PASSAGE DEVICE FOR USE IN A RESPIRATORY MACHINE

(71) Applicant: WALLENBERG UNION LLC, Newark, DE (US)

(72) Inventor: David LUO, Newark, DE (US)

(21) Appl. No.: 18/778,400

(22) Filed: Jul. 19, 2024

(51) Int. Cl. A61M 16/00

(52) U.S. Cl. CPC A61M 16/0066; A61M 16/0003; A61M 2202/0007; A61M 2202/02; A61M 2205/0216; A61M 2205/025; A61M 2205/103; A61M 2205/3331; A61M 2205/42

(57) ABSTRACT

A noise-reducing air passage device for use in respiratory machines, which includes a housing with an inner wall and an outer wall. The housing also has at least one gas inlet and at least one gas outlet, with the space through which the airflow moves from the inlet to the outlet forming the gas passage of the noise-reducing air passage device. The gas passage forms at least one chamber, within which a blower, serving as the core component of the noise-reducing air passage device, is placed. There is an inlet pipe at the gas inlet and an outlet pipe at the gas outlet. Additionally, the gas passage includes no foam.

### TECHNICAL FIELD

This disclosure relates to a noise-reducing air passage device for use in a respiratory machine for improving or treating conditions such as sleep apnea, where a blower as a core component within the gas passage provides power to the noise-reducing air passage device.

### BACKGROUND

Sleep is a critical physiological process essential for maintaining both physical and mental health. However, as night falls, snoring becomes a common occurrence for many, creating noise that not only disrupts the sleep of others sharing the same bed but may also pose potential health risks to the snorer themselves. Data indicates that over 40% of adults snore at least occasionally, with a significant number possibly suffering from conditions such as Obstructive Sleep Apnea (OSA). While not all individuals who snore have OSA, most people who snore are likely to suffer from OSA or OSA-related conditions, including high blood pressure, heart disease, stroke, acid reflux, atrial fibrillation (an irregular heartbeat), depression, and diabetes.

Existing treatment methods for sleep-related breathing disorders are broadly divided into non-surgical and surgical treatments. Non-surgical treatment includes the use of home respiratory machines that deliver gas through the patient's nose or mouth to maintain continuous positive airway pressure. This is currently the most effective non-surgical treatment for sleep apnea. This method prevents the passive collapse of soft tissues during inhalation and stimulates the mechanical receptors in the genioglossus muscle, increasing airway tension and making breathing easier, although it requires long-term, uninterrupted use of the home respiratory machine. Particularly in the United States, most patients achieve satisfactory results with home respiratory machines. In addition to using respiratory machines to treat sleep apnea, patients can also opt to wear various orthotics that elevate the soft palate, actively or passively pull the tongue forward, or directly move the jaw forward during sleep to expand the oropharyngeal and hypopharyngeal spaces to improve breathing. This method is suitable for patients with mild or moderate conditions but is ineffective for severe cases. Oxygen therapy and various pharmacological treatments, such as neuro-respiratory stimulants, also serve as auxiliary treatment methods. For certain special cases, surgery may also be considered. Surgical options include nasal surgery, tongue reduction surgery, uvulopalatopharyngoplasty (UPPP), and orthognathic surgery. The purpose of surgical treatments is to reduce or eliminate airway obstruction and prevent the collapse of airway soft tissues. The choice of surgical method depends on the location and severity of the airway blockage, the presence of pathological obesity, and the overall health condition of the patient. In everyday life, patients can also alleviate symptoms by paying attention to diet, exercising, psychological care, correcting eating and living habits, reducing body weight by 5% to 10% within a specified time, quitting smoking and drinking, avoiding the use of sedatives before sleep, controlling sleep posture, and avoiding sleeping on the back. These lifestyle adjustments can also help relieve symptoms.

Observations show that non-surgical treatment, especially Continuous Positive Airway Pressure (CPAP) therapy, is the most popular and considered the most effective treatment option among various methods. This treatment works by providing a continuous positive airflow to the patient's respiratory tract to prevent airway collapse. Furthermore, automatic sleep apnea treatment devices can adjust the air pressure automatically based on the individual's breathing status during sleep, keeping the upper respiratory tract open in response to the patient's breaths. Additionally, the market offers bi-level and single-level respiratory machines, suitable for patients with mild, moderate, and severe conditions.

### SUMMARY

The objective of this disclosure is to provide a new type of noise-reducing air passage device for use in respiratory machines, which not only achieves noise reduction but also ensures patient health and safety. This is advantageous for the manufacturing of noise-reducing air passage devices and for rapidly adapting them to the market. The foam-free internal structure of the noise-reducing air passage device allows for prolonged and extensive use by patients and overcomes the limitations present in similar existing technologies. Thus, it provides a more effective solution with broader application scenarios and spaces, offering a safer method of delivering a continuous positive airflow to the patient's airway for the treatment of sleep-related breathing disorders.

This disclosure provides a noise-reducing air passage device for use in a respiratory machine, which is configured to pressurize gas and provide the pressurized gas to a patient's airway. The noise-reducing air passage device includes:
a blower, which includes an intake port and an exhaust port and is configured to generate a flow of the pressurized gas;
a housing, which includes at least one gas inlet to receive breathable gas, at least one gas outlet to allow the pressurize gas to flow out, an inner wall, and an outer wall;
and a gas passage, which is a space surrounded by the inner wall of the housing, and is configured to allow the breathable gas to flow through. The gas passage forms at least one chamber within the inner wall, and a total volume of the at least one chamber is more than three times a volume of the blower.

The outlet pipe is provided at the at least one gas outlet, and the outlet pipe is configured to connect to the at least one gas outlet and communicate with the exhaust port of the blower. In addition, the gas passage does not include foam.

In one embodiment, the gas passage forms a first chamber and a second chamber.

In one embodiment, a volume of the first chamber is greater than a volume of the second chamber.

In one embodiment, an axis of the gas inlet is parallel or perpendicular to an axis of the intake port of the blower.

In one embodiment, an inlet pipe is provided at the gas inlet, which is configured to connect to the housing and include a taper.

In one embodiment, the housing forms a part of the respiratory machine.

In one embodiment, the housing of the noise-reducing air passage device includes one of the following materials: polypropylene, polycarbonate, polyethylene terephthalate glycol-modified-1,4-cyclohexanedimethanol ester, polyamide, or polyether ether ketone.

This disclosure also provides a noise-reducing air passage device for use in a respiratory machine, which is configured to pressurize gas and provide the pressurized gas to a patient's airway. The noise-reducing air passage device includes:
a blower, which includes an intake port and an exhaust port and is configured to generate a flow of the pressurized gas;
a housing, which includes at least one gas inlet to receive breathable gas, at least one gas outlet to allow the pressurize gas to flow out, an inner wall, and an outer wall;
and a gas passage, which is a space surrounded by the inner wall of the housing, and is configured to allow the breathable gas to flow through. The gas passage forms at least one chamber within the inner wall, and a total volume of the at least one chamber is more than three times a volume of the blower;

The outlet pipe is provided at the gas outlet, and the outlet pipe is configured to connect to the at least one gas outlet and communicate with the exhaust port of the blower. The outlet pipe has at least one of the following characteristics:
a. the outlet pipe including at least one section of a wall that is coaxial with an axis of the exhaust port of the blower;
b. a length of the outlet pipe being equal to or greater than a diameter of the exhaust port of the blower; and
c. an opening area of the outlet pipe being between 75% to 125% of an area of the exhaust port of the blower.

And the gas passage does not include foam.

In one embodiment, the outlet pipe includes at least one section of the wall near the exhaust port of the blower that is coaxial with the axis of the exhaust port of the blower, and a length of the at least one section of the wall is at least 6 mm.

In one embodiment, an inlet pipe is provided at the at least one gas inlet.

In one embodiment, distances from a center of the intake port of the blower to four sides of the inner wall of the housing are approximately equal.

In one embodiment, the housing has a section of the wall near the intake port of the blower that is substantially coaxial with the intake port of the blower.

In one embodiment, the outlet pipe includes one of the following materials: plastic, silicone, rubber, thermoplastic elastomer, thermoplastic polyurethane, or fluororubber.

In one embodiment, the housing forms a part of the respiratory machine.

This disclosure further provides a noise-reducing air passage device for use in a respiratory machine, which is configured to pressurize gas and provide the pressurized gas to a patient's airway. The noise-reducing air passage device comprises:
a blower, which includes an intake port and an exhaust port and is configured to generate a flow of the pressurized gas;
a housing, which includes at least one gas inlet to receive breathable gas, at least one gas outlet to allow the pressurized gas to flow out, an inner wall, and an outer wall;
and a gas passage, which is a space surrounded by the inner wall of the housing, and is configured to allow the breathable gas to flow through. The gas passage forms at least one chamber within the inner wall, and a total volume of the at least one chamber is more than three times a volume of the blower.

An inlet pipe is provided at the at least one gas inlet, and is configured to include an intake end connectable to the housing and an outlet end to discharge the breathable gas. The inlet pipe has at least one of the following characteristics:
a. a straight-line distance from a center of the outlet end of the inlet pipe to a center of the intake port of the blower being between 35 mm to 150 mm; and
b. a ratio of a length of the inlet pipe to the straight-line distance from the center of the outlet end of the inlet pipe to the center of the intake port of the blower being between 0.16 to 2.29.

A distance between the intake port of the blower and the opposing inner wall of the housing is greater than 5 mm, and the gas passage does not include foam.

In one embodiment, the inlet pipe is provided at an edge portion of the gas passage.

In one embodiment, a transitional component is provided between the gas outlet and the exhaust port of the blower that connects the two and prevents gas leakage.

In one embodiment, the noise-reducing air passage device includes a first chamber, a second chamber, and a wall that isolates the first chamber from the second chamber, and the wall includes an opening and is configured to communicate with the intake port of the blower.

In one embodiment, an axis of the gas outlet is not located on a horizontal plane of an axis of the inlet pipe.

In one embodiment, the noise-reducing air passage device does not contain any components made from polymer foaming materials.

In one embodiment, the housing forms a part of the respiratory machine.

This disclosure further discloses a noise-reducing air passage device for use in a respiratory machine, which is configured to pressurize gas and provide the pressurized gas to a patient's airway. The noise-reducing air passage device comprises:
a blower, which includes an intake port and an exhaust port, and is configured to generate a flow of the pressurized gas;
a housing, which includes at least one gas inlet to receive breathable gas, at least one gas outlet to allow the pressurized gas to flow out, an inner wall, and an outer wall;
and a gas passage, which is a space surrounded by the inner wall of the housing, configured to allow the pressurized gas to flow through. The gas passage forms at least one chamber within the inner wall.

An inlet pipe is provided at the at least one gas inlet, which is configured to include an intake end connectable to the housing and an outlet end to discharge the breathable gas. An area enclosed by a wall around the intake end of the inlet pipe is not less than 75% of an area of the intake port of the blower.

The gas inlet and gas outlet are not on a same wall, and the gas passage does not include foam.

In one embodiment, the intake end of the inlet pipe includes a trumpet-shaped elastomer, which is configured to smoothly guide gas into the chamber.

In one embodiment, a distance between the intake port of the blower and the opposing inner wall of the housing is greater than 5 mm.

In one embodiment, a distance between the outlet end of the inlet pipe and the opposing inner wall of the housing is greater than 1.5 times a diameter of the intake end of the inlet pipe.

In one embodiment, the inlet pipe has a draft angle, and the draft angle is at least 1.50.

In one embodiment, the gas inlet and the gas outlet are non-coaxial.

In one embodiment, the housing forms a part of the respiratory machine.

Implementing the noise-reducing air passage device of this disclosure discussed herein provides at least the following benefits:

1) The design of a foam-free noise-reducing air passage device enhances the safety of respiratory machines (such as ventilators). In 2021, a globally renowned brand issued its first worldwide recall notice involving some of its Bi-level Positive Airway Pressure (BiPAP) devices, Continuous Positive Airway Pressure (CPAP) devices, and mechanical ventilators, followed by several more recalls. The recalls were primarily due to the use of sound-dampening foam in the noise-reducing air passage device, which could release particles and volatile organic compounds. In 2023, the FDA received thousands of complaints about this internationally known brand's CPAP and BiPAP machines. This incident had a significant negative impact on the brand's reputation.

The FDA requires that ventilators must demonstrate a noise level below 30 dB for market approval. Using foam for noise reduction is currently the simplest method because foam materials are easily obtainable and manufacturable. Their unique porous structure and material properties can convert noise into minimal energy, thereby reducing noise. Indeed, using foam to reduce noise can achieve a good noise reduction effect, and placing foam inside the noise-reducing air passage device is the simplest, most effective, and common means to meet regulatory noise standards.

Therefore, nearly all respiratory machines on the current market incorporate foam within the gas passage for noise reduction. However, foam can easily cause health issues for several reasons:

A. Due to the softness and relatively loose surface of foam materials, they can be easily worn away or peeled off by airflow during use, releasing particles. Once released, these particles can easily enter the patient's airway with the airflow, irritating the respiratory system. This may cause respiratory problems, leading to symptoms such as sore throat and coughing, especially in individuals already suffering from respiratory diseases such as asthma or Chronic Obstructive Pulmonary Disease (COPD).

B. Additionally, foam is often made from synthetic materials that may contain residual chemical additives. These chemicals can gradually be released as the foam ages and degrades. In some cases, if foam particles carry harmful microbes, they could lead to potential infections, particularly in individuals with weakened immune systems. Foam particles may also trigger allergic reactions, including sneezing, flu-like symptoms, and eye irritation.

C. Furthermore, foam used over long periods can accumulate dust, bacteria, and other contaminants, especially in respiratory machines. The device can easily inhale contaminants from the air, leading to bacterial growth and increasing the risk of infection.

This disclosure discussed herein specifically focuses on the safety and reliability of the ventilator's noise-reducing air passage device during design, implementing a series of stringent safety measures, including a foam-free design of the noise-reducing air passage device to reduce potential health risks to patients using ventilators. In designing the gas passage and filtration system, foam-free or easily replaceable foam designs are used to mitigate these potential health risks. For patient health and safety, the gas passage is designed to be foam-free. Since there is no foam in the gas passage, it reduces the chance of accumulating minute foreign objects, helping to maintain cleanliness in the gas passage. More importantly, the air breathed is not affected by minute residues from the foam itself, lowering the number of particles that patients might inhale or come into contact with, ensuring safety during device use. This is particularly important for patients who use the device over a long period as it helps to reduce potential respiratory issues. Additionally, some patients may be allergic to particles from materials such as foam, and the foam-free design reduces the risk associated with allergic reactions. This is also crucial for those allergic to foam materials or sensitive to chemically treated materials. Through multiple tests, this product has demonstrated that the foam-free noise-reducing air passage device can enhance patient safety and comfort during device use.

2) The design of the foam-free noise-reducing air passage device enhances reliability and extends the lifespan of the device. Foam is typically made from synthetic materials like polyurethane and polyether, which are softer compared to plastics and silicone and more susceptible to mechanical damage and chemical erosion. Foam is highly prone to aging due to environmental factors and often contains chemical additives or components that can alter its performance. These characteristics result in a shorter lifespan for foam materials, theoretically necessitating frequent replacements to maintain their effectiveness in noise reduction within the air passage device. In contrast, materials such as silicone and plastic usually have better wear resistance, corrosion resistance, and durability. They are less likely to be affected by environmental conditions and generally have better chemical stability, making them less susceptible to chemical factors and thus longer-lasting. The foam-free design of the noise-reducing air passage device eliminates foam, thereby extending the lifespan of the noise-reducing air passage device and, consequently, the device itself. Additionally, the foam-free design simplifies the internal structure of the device's gas passage by eliminating the need for extra components to secure foam, reducing the number of components and thus lowering manufacturing complexity. This helps enhance the device's reliability and stability. Moreover, foam materials often require timely replacement and cleaning to ensure health, but foam placed inside devices cannot usually be replaced or cleaned. The foam-free design avoids these steps, reducing the maintenance needs within the gas passage and increasing the convenience of using the device.

3) By employing effective noise-reducing structures, the noise-reducing air passage can achieve regulatory noise levels even without foam assistance. This noise-reducing air passage device utilizes various structures that effectively reduce noise, replacing the traditional role of foam within the air passage device. Specifically, the disclosure discussed herein employs multiple noise-reducing structures including, but not limited to, a conical inlet pipe, a trumpet-shaped elastomer made from flexible materials, a more reasonably placed blower, an arc-shaped wall that forms the airflow entering the intake port of the blower and is essentially coaxial with the blower inlet, and curved corners on the inner wall of the gas passage that correspond with the airflow path. The use of these structures and components not only effectively lowers noise levels but also enhances the stability and reliability of the noise-reducing air passage device. More importantly, the noise-reducing components within the air passage device, the internal structure of the air passage device, and the relationships among various structures are all based on substantial experimental data support and scientific analysis, ensuring the scientific validity and credibility of the noise-reducing air passage design. This scientifically credible noise-reducing air passage device design not only enhances the product's performance but also provides patients with a quieter and more reliable user experience.

4) The disclosure discussed herein reduces the cost of the device and aligns with environmental principles by using simpler materials and structures. The design of the noise-reducing air passage device in this disclosure discards traditional foam materials, utilizing only plastic components for the housing of the noise-reducing air passage device and as materials for forming chambers and passages, and silicone materials for a fixed connection between the blower and the housing of the noise-reducing air passage device. Compared to the more complex foam-containing structures in ventilators' noise-reducing air passage devices available on the market, this design, comprised of only two materials, is easier to process and assemble, making its manufacturing process simpler and more efficient. Additionally, by reducing the use of auxiliary materials like foam, it also lessens the environmental impact, aligning with modern society's environmental requirements and trends and making a positive contribution to environmental protection. Since foam releases harmful chemicals during production and disposal, which pollute the environment, the foam-free design of the noise-reducing air passage device avoids the release of these harmful substances, reducing negative environmental impacts. Therefore, this foam-free design using only silicone and plastic not only reduces the cost of the device but also adheres to ecological principles, representing a more sustainable and economical design solution.

### BRIEF DESCRIPTION OF THE DRAWINGS

FIG. 1 shows a three-dimensional schematic diagram of the internal structure of a noise-reducing air passage device according to an embodiment of the present disclosure;

FIG. 2 shows a three-dimensional schematic diagram of the housing of a noise-reducing air passage device according to an embodiment of the present disclosure;

FIG. 3 shows a top view of the internal structure of a noise-reducing air passage device according to an embodiment of the present disclosure;

FIG. 4 shows an exploded view of the housing of a noise-reducing air passage device according to an embodiment of the present disclosure;

FIG. 5 shows a three-dimensional schematic diagram of the blower of a noise-reducing air passage device according to an embodiment of the present disclosure;

FIG. 6 shows a three-dimensional schematic diagram of the transitional component of a noise-reducing air passage device according to an embodiment of the present disclosure;

FIG. 7 shows an exploded view of the structure of a noise-reducing air passage device according to an embodiment of the present disclosure;

FIG. 8 shows a diagram of the airflow path of a noise-reducing air passage device according to an embodiment of the present disclosure;

FIG. 9 shows a schematic diagram of multi-angle, repeated testing of a noise-reducing air passage device according to an embodiment of the present disclosure.

FIG. 10 shows a schematic diagram of the length of the inlet pipe and the distance from the center of the outlet end of the inlet pipe to the center of the intake port of the blower according to an embodiment of the present disclosure;

FIG. 11 shows a schematic diagram of the distance from the outlet end of the inlet pipe to the wall according to an embodiment of the present disclosure;

FIG. 12 shows a schematic diagram of the taper of the inlet pipe according to an embodiment of the present disclosure;

FIG. 13 shows a schematic diagram of the airflow path before entering the blower according to an embodiment of the present disclosure;

FIGS. 14A and 14B show schematic diagrams where the distances from the intake port of the blower to the surrounding walls are essentially the same, according to an embodiment of the present disclosure;

FIG. 15 shows a schematic diagram where the inlet pipe and the housing are not integrally formed according to an embodiment of the present disclosure;

FIG. 16 shows a three-dimensional schematic diagram of the inlet pipe provided within the chamber in an embodiment of the present disclosure;

FIG. 17 shows a three-dimensional schematic diagram of a noise-reducing air passage device without an inlet pipe in an embodiment of the present disclosure;

FIG. 18 shows a three-dimensional schematic diagram of a noise-reducing air passage device with a different internal structure according to an embodiment of the present disclosure;

FIG. 19 shows a three-dimensional schematic diagram of another noise-reducing air passage device with a different internal structure according to an embodiment of the present disclosure;

FIG. 20 shows a three-dimensional schematic diagram of yet another noise-reducing air passage device with a different internal structure according to an embodiment of the present disclosure;

FIG. 21 shows a three-dimensional schematic diagram of a chamber with protruding guide vanes or external baffles forming a wall essentially coaxial with the intake port of the blower, according to an embodiment of the present disclosure;

FIG. 22 shows a three-dimensional schematic diagram of a noise-reducing air passage device with a trumpet-shaped elastomer at the gas inlet, according to an embodiment of the present disclosure;

FIG. 23 shows a three-dimensional schematic diagram of a noise-reducing air passage device containing soundproofing materials other than foam, according to an embodiment of the present disclosure;

FIG. 24 shows a schematic diagram analyzing the airflow direction inside a noise-reducing air passage device, according to an embodiment of the present disclosure;

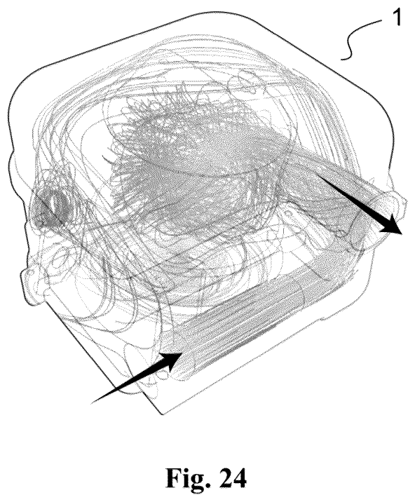

FIG. 25 shows a three-dimensional schematic diagram of the housing of a noise-reducing air passage device forming a part of a respiratory machine, according to an embodiment of the present disclosure;

FIG. 26 shows a sectional view of the housing of a noise-reducing air passage device forming a part of a respiratory machine, according to an embodiment of the present disclosure.

### DETAILED DESCRIPTION

To facilitate a better understanding of the disclosure, a more comprehensive description will be provided with reference to the accompanying drawings that depict typical embodiments of the disclosure. However, the disclosure can be implemented in many different forms and should not be construed as limited to the embodiments set forth herein. Instead, these embodiments are provided to ensure a thorough and complete understanding of the disclosure.

Unless defined otherwise, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

This disclosure addresses the existing market use of foam for noise reduction in air passages for ventilators, which is prone to damage and aging, detrimental to patient health and safety, involves more complex manufacturing processes, and is not environmentally friendly. The disclosure discussed herein provides a safer, more reliable, and simpler design for noise-reducing air passage devices. The noise-reducing air passage device discussed in this disclosure not only optimizes various disadvantages of the existing designs but also achieves the regulatory noise levels, making it a superior technical design for patients, producers, and the market. Choosing not to use foam within the noise-reducing air passage device also represents a sustainable and environmentally friendly design approach.

The following specific examples illustrate several structures of the noise-reducing air passage device for ventilators as applied in this disclosure.

Clarifications of terms used in the embodiments are provided as follows:

“Basic,” “approximately,” and “roughly”: In some forms of this technology, these terms indicate a variation within fifteen percent above or below the original numerical value.

“Air/Gas”: In some forms of this technology, air/gas is considered to be the air/gas used for breathing in everyday life. In other forms of this technology, air/gas may be considered to refer to other gases or combinations of gases suitable for breathing, such as atmospheres enriched with a higher oxygen content.

“Environment”: In some forms of this technology, the environment may be considered to refer to the external area outside the housing of the noise-reducing air passage device that does not enclose the blower. In other forms of this technology, the environment is considered to be the surroundings of the patient's location.

### Embodiment 1

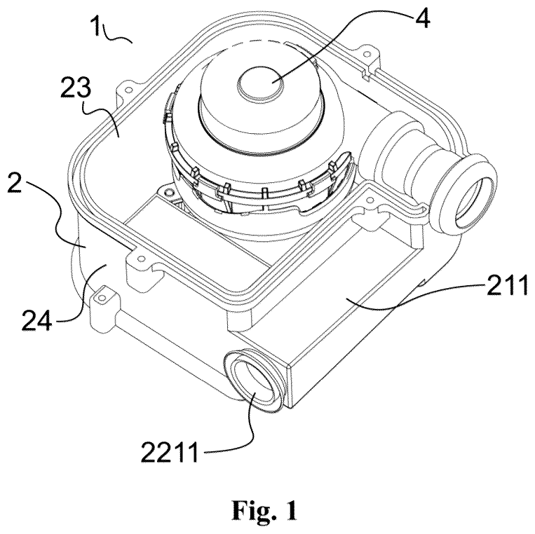

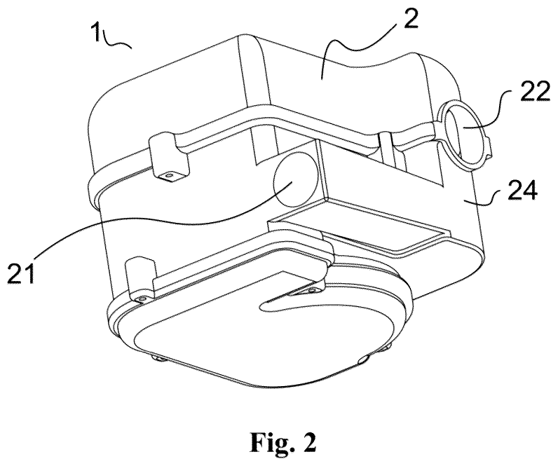

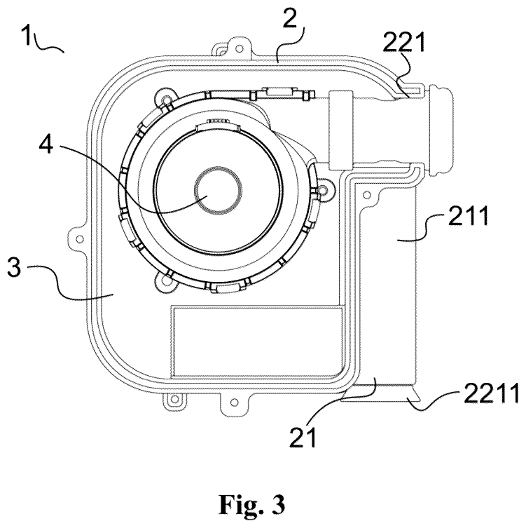

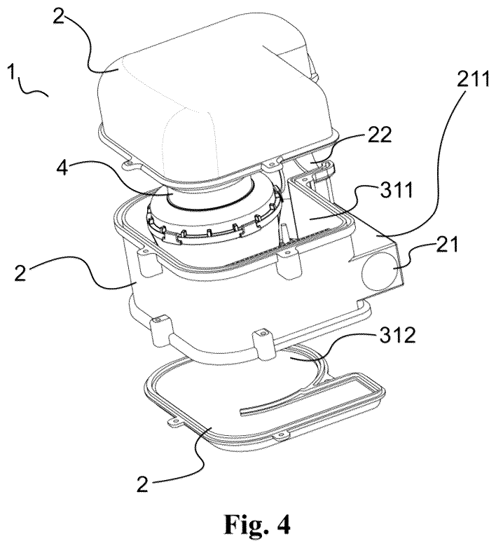

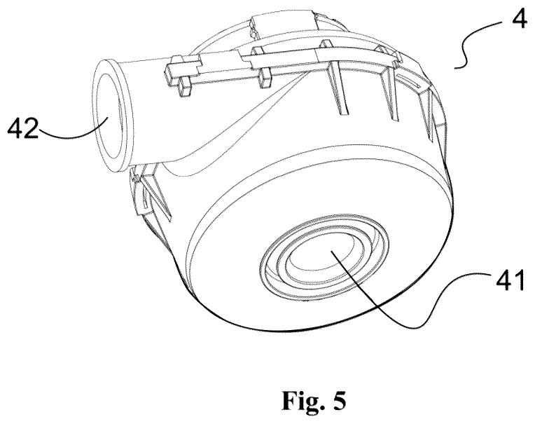

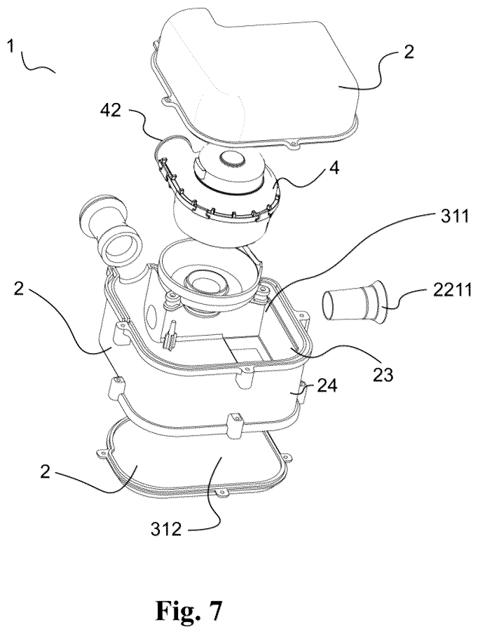

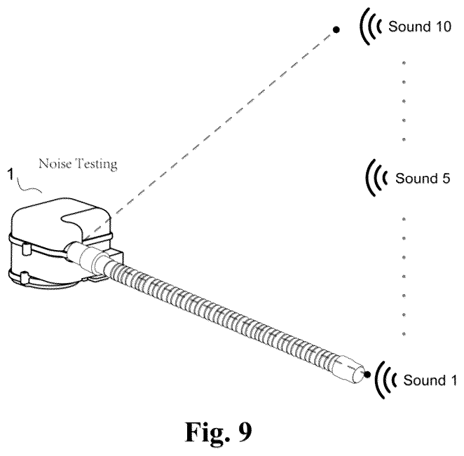

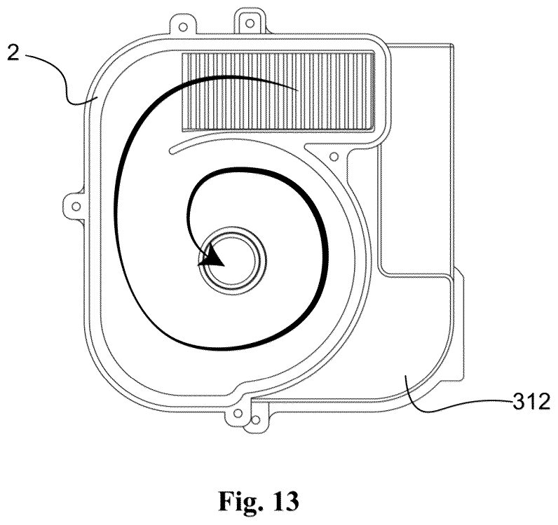

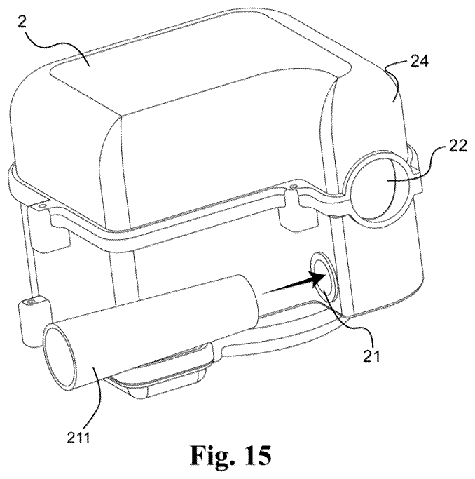

This embodiment provides a noise-reducing air passage device 1, applied in ventilators. This embodiment offers three-dimensional schematic diagrams, top views, exploded and disassembled diagrams, airflow path diagrams, test scenario images, and various data illustrations for noise-reducing air passage device 1, as referenced in FIGS. 1-15. This embodiment involves a noise-reducing air passage device 1 used in respiratory machines. It is configured to pressurize gas and supply the pressurized gas to the patient's airway. The noise-reducing air passage device 1 includes a housing 2 with an inner wall 23 and an outer wall 24, a gas inlet 21 and a gas outlet 22 on the housing 2, a foam-free gas passage 3, chambers 31, a blower 4, an inlet pipe 211, and an outlet pipe 221. It is designed to integrate with other components, such as a circuit board, to form a complete respiratory system treatment device. The system is used to blow the pressurized gas from the blower 4 into the patient's airway in a controlled manner, thus preventing the collapse of the patient's airway.

The noise-reducing air passage device 1 includes a blower 4 with an intake port 41 and an exhaust port 42, configured to generate a flow of pressurized gas. The blower 4 is positioned within the gas passage 3 in such a way that the plane of its intake port 41 may be parallel or perpendicular to the horizontal plane. In this embodiment, the blower 4 is approximately located at the central position of the entire gas passage 3, which aids in balancing the noise-reducing air passage device 1. Additionally, the blower 4 can also be positioned near the edge of one of the walls of the housing 2.

The noise-reducing air passage device 1 also includes a housing 2 that features at least one gas inlet 21 for receiving breathable gas, at least one gas outlet 22 for allowing the pressurized gas to flow out, an inner wall 23, and an outer wall 24. The gas inlet 21 on the housing 2 is configured to draw breathable gas from the external environment to provide breathable gas for pressurization by the blower 4. One end of the gas outlet 22 is connected to the exhaust port 42 of the blower 4, while the other end connects to a hose or a passage on the housing 2. The gas inlet 21 and the gas outlet 22 are not on the same wall, and the axis of the gas outlet 22 is not on the same horizontal plane as the axis of the inlet pipe 211, meaning there is a vertical difference in height between the gas outlet 22 and the inlet pipe 211 (the gas inlet 21 and the gas outlet 22 being at different vertical heights). This setup helps to provide a quieter environment for respiratory machines. Due to the noise generated by both the suction and gas delivery of the blower 4, where the noise at the intake port 41 of the blower 4 is greater than at the exhaust port 42, placing the gas inlet 21 and gas outlet 22 on two different walls of the housing 2 effectively reduces the noise transmitted from the gas inlet 21, keeps the noise as far away from the patient as possible, and prevents the overlapping of noise from the intake port 41 and the exhaust port 42. In one arrangement, the gas inlet 21 and the gas outlet 22 are non-coaxial, which also helps keep the patient away from the noise source and reduces the noise heard by the patient. If the gas passage 3 forms at least two chambers 31, the housing 2 of the noise-reducing air passage device 1 also includes a wall that separates these chambers 31.

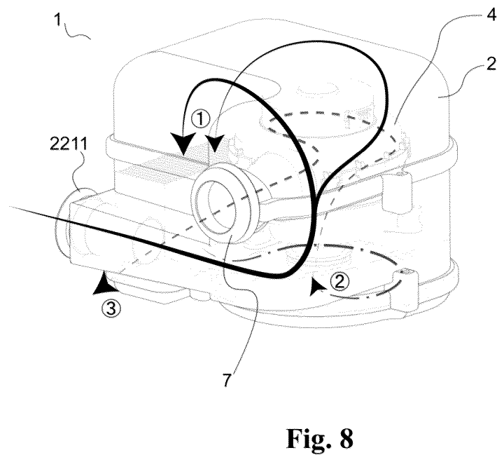

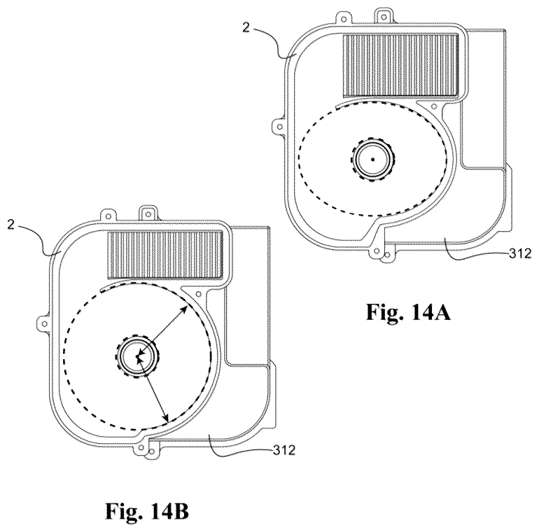

The gas passage 3 is a space surrounded by the inner wall 23 of the housing 2, and is configured to allow breathable gas to flow through. The gas passage 3 forms at least one chamber 31 within the inner wall 23. In some implementations, there are at least two chambers 31, specifically forming a first chamber 311 and a second chamber 312 in the gas passage 3. Larger volumes of the chambers 31 generally result in lower resistance to the movement of breathable gas, thereby reducing the likelihood of turbulence. However, when the volume of the gas passage 3 is excessively large, the increased volume of the chambers 31 creates more dead space, hindering effective gas circulation, thus reducing gas flow rate and increasing noise. Extensive testing of various volume ratios between the chambers 31 and the blower 4 has concluded that the total volume of the chambers 31 being more than three times the volume of the blower 4, and preferably between 3 to 16 times, results in a more noise-reducing form of the gas passage 3. Additionally, when the gas passage 3 includes at least two chambers 31, the noise-reducing air passage device 1 comprises the first chamber 311, the second chamber 312, and a wall that isolates the first chamber 311 from the second chamber 312, with an opening on this wall configured to connect to the intake port 41 of the blower 4. The volume of the first chamber 311 is greater than that of the second chamber 312. More specifically, the blower 4 is located in the first chamber 311 with the intake port 41 of the blower 4 connected to the second chamber 312. To ensure sufficient space in the second chamber 312 for smooth gas entry into the blower 4, the intake port 41 of the blower 4 is positioned at a distance greater than 4 mm from the wall separating the chambers 31 within the noise-reducing air passage device 1, and the distance between the intake port 41 of the blower 4 and the opposing inner wall 23 of the housing 2 is set to be more than 5 mm, preferably 10 mm. Due to vibrations produced by blower 4 during operation, when these vibrations are transmitted to the housing 2 of noise-reducing air passage device 1, the rigid material of the housing 2 does not absorb or alleviate the vibrations, resulting in significant noise. Therefore, in some cases, a flexible material such as silicone may be used at the opening to prevent vibrations from directly reaching the housing 2 of the noise-reducing air passage device 1, thus achieving vibration damping and noise reduction. Before the air enters the blower 4 from the chamber 31, it typically follows a primary flow path. The direction of the airflow entering the blower 4 is set to align with the rotation direction of the blower's impeller, which effectively reduces the impact of the airflow, thereby making the noise-reducing air passage device 1 quieter. The placement of the blower 4 within the noise-reducing air passage device 1 also has specific requirements. The design ensures that the distances from the center of the intake port 41 of the blower 4 to four sides of the inner wall 23 of the housing 2 are approximately equal (as shown in FIGS. 14A and 14B), positioning the intake port 41 of the blower 4 at the approximate center of its chamber 31, which facilitates uniform airflow through the opening into the blower 4. The inner wall of the gas passage 3 that face the airflow path are rounded, meaning that the parts where the two sides of the inner wall 23 of the housing 2 meet have rounded corners, giving the inside of the gas passage 3 a more rounded overall shape. This design avoids intense collisions of airflow with the wall inside the gas passage 3 and also guides the direction of the airflow. The airflow path within the entire gas passage 3 starts by entering the chamber 31 through the inlet pipe 211, following the flow path set within the chamber 31. In some cases, it includes entering the second chamber 312 from the first chamber 311 through an opening of the second chamber 311, then entering the blower 4 through the opening, and after pressurization by the blower 4, reaching the outlet pipe 221 through the exhaust port 42 of the blower 4. From a top view, this airflow path includes at least one segment with a staggered configuration, and in some instances, the axis of the gas inlet 21 is parallel or perpendicular to the axis of the intake port 41 of the blower, forming a spatial airflow path within the noise-reducing air passage device 1. Specifically, the airflow path formed by the gas inlet 21, the chamber 31, the blower 4, and gas outlet 22 extends in space rather than merely on the same plane, meaning the airflow in gas passage 3 has at least one segment that flows vertically (as shown in FIG. 8). Additionally, the gas passage 3 includes no foam; here, ‘foam’ refers to foam materials made from polymer foaming materials, including one or more of the following materials: polyurethane foam, polyester foam, polyether foam, neoprene, cross-linked polyethylene, or those containing polyvinyl chloride (PVC), polyetherimide (PEI), and styrene acrylonitrile (SAN or AS), polymethyl methacrylimide (PMI), foamed polyester (PET). The housing 2 of the noise-reducing air passage device 1 is made from one of the following materials: polypropylene (PP), polycarbonate (PC), polyethylene terephthalate glycol-modified-1,4-cyclohexanedimethanol ester (PCTG), polyamide (PA), or polyether ether ketone (PEEK).

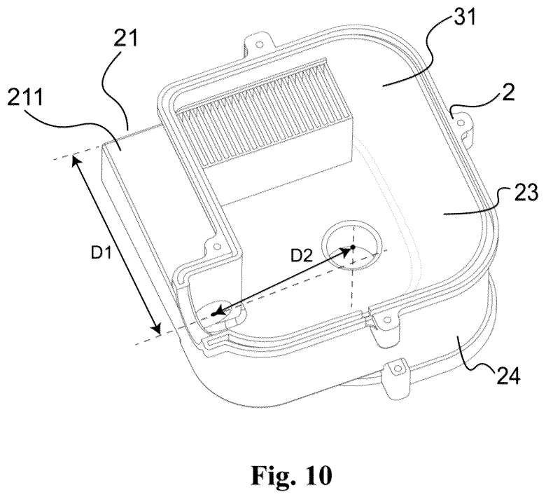

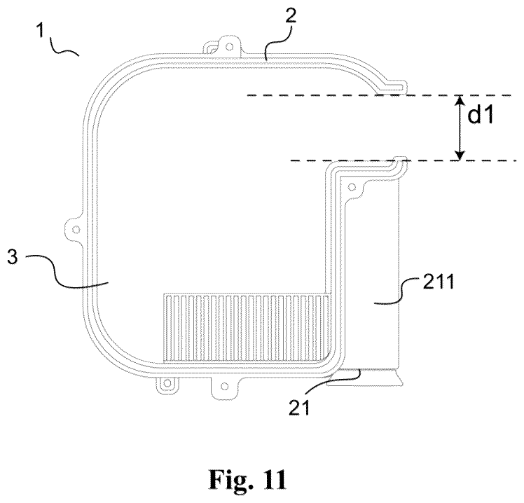

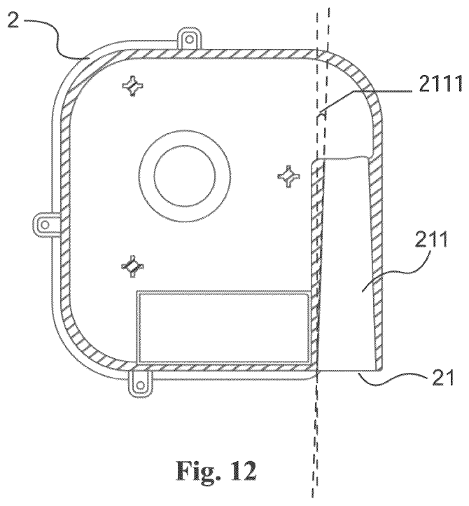

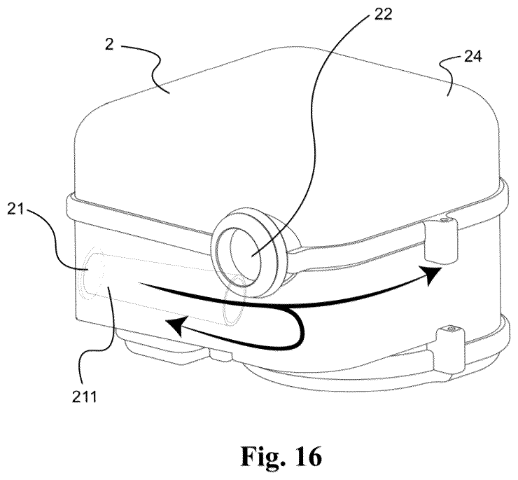

The gas inlet 21 includes an inlet pipe 211 and an outlet pipe 221 that is connected to the gas outlet 22 and communicates with the exhaust port 42 of the blower 4. The inlet pipe 211 is configured to have an intake end to connect to the housing 2 and an outlet end that releases the breathable gas. Both the inlet pipe 211 and the outlet pipe 221 are configured to attach to the housing 2, and their connections with the housing 2 can either be integrated with or detached from the housing 2. Typically, the cross-section of the inlet pipe 211 is concentrically circular, although in some cases, it may also be square, hexagonal, or another appropriate shape. The inlet pipe 211 is configured to connect to the housing 2 and forms a path for breathable gas from the external environment into the interior of the gas passage 3. The inlet pipe 211 is positioned at the edge of the gas passage 3 (on the outer side away from the center of the noise-reducing air passage device) and isolated from the internal chamber 31, which helps prevent unnecessary breathable gas flow within the gas passage 3, such as avoiding breathable gas from flowing into the tiny gaps between the inlet pipe 211 and the inner wall 23 of the housing 2, thereby causing noise (as shown in FIG. 16). To effectively gather and streamline the airflow entering the noise-reducing air passage device 1 through the inlet pipe 211, its length is specified. From the previous discussion, the optimal volume of the gas passage 3 is 3-16 times the volume of the blower 4; hence, the length of the inlet pipe is calculated based on the three times and sixteen times volume of the gas passage 3. For the circular cross-section of the inlet pipe 211, the pressure gradient of the fluid can be calculated using the formula 1=ρL/A, where L is the length of the inlet pipe 211, ρ is the density of air, and A is the cross-sectional area of inlet pipe 211. This method allows for estimating the noise based on gas pressure, and the inlet pipe 211 can be as long as possible, ranging from 25 mm to 80 mm (as shown in FIG. 10, D1) without touching the wall of housing 2 or obstructing its outlet end. To ensure that airflows do not interfere with each other, the distance from the outlet end of the inlet pipe 211 to the intake port 41 of the blower 4 is also specified. Calculate the distance from the center of the outlet end 42 of the inlet pipe 211 to the center of the intake port 41 of the blower 4 (as shown as D2 in FIG. 10), using the volume of the gas passage 3 which is 16 times the volume of the blower 4: Calculating the volume of the gas passage 3 as sixteen times the volume of the blower 4 gives 2,118,928 mm³, with each side averaging 128.4 mm in length. Assuming both the blower 4 and the inlet pipe 211 are positioned at the outermost edges, the volume of the blower is fixed, and the center of the blower's intake port 41 is 8.4 mm from the top, hence the vertical distance between the center of the blower's intake port 41 and the center of the outlet end of the inlet pipe 211 is the total length minus the distance from the top, which is 120 mm. The center of the blower's intake port 41 is 38.4 mm from the side wall, and thus the horizontal distance between the center of the blower's intake port 41 and the center of the outlet end of the inlet pipe 211 is the total length minus the distance to the side, which is 90 mm. Finally, using the Pythagorean theorem, the actual distance between the center of the blower's intake port 41 and the center of the outlet end of the inlet pipe 211 is calculated to be 150 mm. Similarly, calculations based on the three times volume edge value yield a Fig. of 35 mm, giving a range for the straight-line distance from the center of the outlet end of inlet pipe 211 to the center of the intake port 41 of the blower 4 as between 35 mm to 150 mm. From the calculated ranges of the inlet pipe 211 length and the distance from the center of its outlet end to the center of the blower's intake port 41, the ratio of the length of the inlet pipe 211 to the distance from the center of its outlet end to the center of the blower's intake port 41 ranges from 0.16 to 2.29. Additionally, under general conditions, the gas inlet 21 features an inlet pipe 211, which is configured to connect to the housing 2 and has a taper 2111. The inlet pipe 211 has a draft angle greater than or equal to 1.5°, preferably between 1.5° to 3° (as shown in FIG. 12, La 2111). The design of this taper 2111 reduces the occurrence of turbulence and vortices within the gas passage 3, facilitating smoother gas flow within noise-reducing air passage device 1. Moreover, the tapering design of the inlet pipe 211 from wide to narrow accelerates the airflow within the pipe, reducing the time air remains within the inlet pipe 211, thus preventing excessive pressure build-up inside the pipe that could cause increased noise. To ensure that the breathable gas does not exert excessive pressure on the wall of the passage after entering the gas passage 3 through the inlet pipe 211, the distance from the outlet end of the inlet pipe 211 to the opposing inner wall 23 of the housing 2 is configured to be greater than 1.5 times the diameter of its intake end (as shown in FIG. 11, d1). For example, if the diameter of the inlet pipe 211 is 16 mm, then the outlet end of the inlet pipe 211 should be at least 24 mm away from the inner wall 23 of the housing 2. Furthermore, to ensure a normal air supply, requirements have been set for the area of the intake end of the inlet pipe 211; the area surrounded by the wall at the intake end of the inlet pipe 211 should not be less than 75% of the area of the blower's intake port 41.

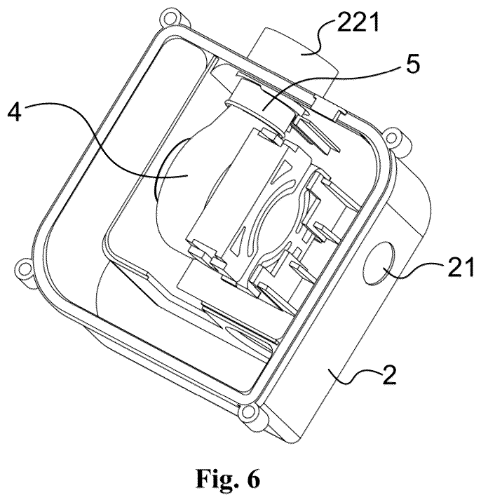

The gas outlet 22 includes an outlet pipe 221, configured to connect to the gas outlet 22 and to communicate (directly or indirectly) with the exhaust port 42 of the blower 4. In one implementation, there is a transitional component 5 between the gas outlet 22 and the exhaust port 42 of the blower 4 that connects them and prevents air leaks (as shown in FIG. 6). The outlet pipe 221 has at least one section (i.e., one or multiple sections) of wall that is coaxial with the axis of the exhaust port 42 of the blower 4, and the length of the outlet pipe 221 is equal to or greater than the diameter of the blower's exhaust port 42, preferably more than 1.5 times. The outlet pipe 221 may have an internal wall that is parallel with the axis of the blower's exhaust port 42; or it may be composed of at least three parts, specifically with at least two sections of the inner wall that are parallel with the axis of the blower's exhaust port 42, and an intermediate section whose internal wall is oriented differently from the axis of the blower's exhaust port 42 (i.e., the axes intersect). Near the exhaust port 42 of the blower 4, the outlet pipe 221 has a section of wall that is coaxial with the axis of the blower's exhaust port 42, and its length is at least 6 mm. Additionally, to ensure the effectiveness of noise-reducing air passage device 1, the opening area of the outlet pipe 221 is set to be 75%-125% of the area of the blower's exhaust port 42, preferably 85%-110%, to ensure that the airflow emitted by the blower 4 does not undergo abrupt changes in speed and flows more smoothly, optimally choosing within the range between 100% to 110%. The outlet pipe 221 is made from one of the following materials: plastic, silicone, rubber, TPE, TPU, or fluoroelastomer.

In another implementation, the gas inlet 21 and the gas outlet 22 are positioned on the same horizontal plane.

In another implementation, the inlet pipe 211 is located within the gas passage 3 and not isolated from the internal chamber 31 (as shown in FIG. 16).

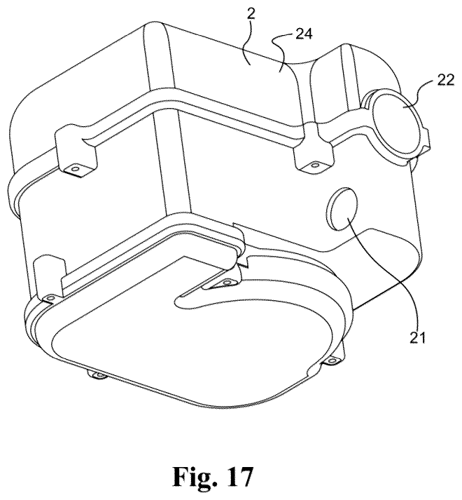

In another implementation, the housing 2 of the noise-reducing air passage device 1 has only a gas inlet 21 with no inlet pipe 211 connected to it (as shown in FIG. 17).

In another implementation, the taper of the inlet pipe 211 can be the basic draft taper required for demolding or have no taper.

In another implementation, the path of the breathable gas through the inlet pipe 211 and the path of the breathable gas entering the blower 4 are neither parallel nor perpendicular.

### Embodiment 2

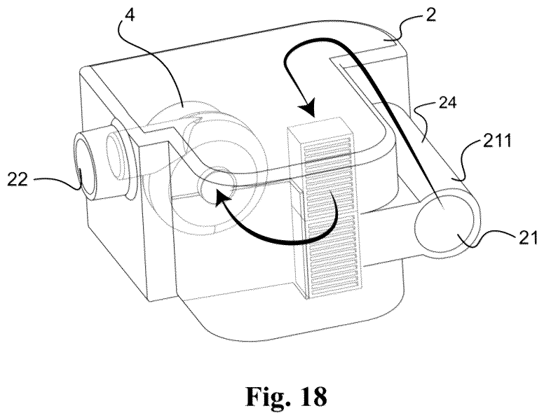

This embodiment provides a noise-reducing air passage device 1 applied in the field of ventilators, referenced in FIG. 18. This embodiment provides a three-dimensional schematic diagram of the noise-reducing air passage device 1. In the embodiment shown in FIG. 18, the difference from Embodiment 1 lies in the placement of the blower 4 within the noise-reducing air passage device 1. Specifically, the blower 4 can be positioned within the noise-reducing air passage device 1 such that the axis of its intake port 41 is perpendicular to the horizontal plane, or parallel to the horizontal plane, and the positional relationship between the first chamber 311 and the second chamber 312 of the gas passage 3 can be arranged either one above the other or on the same horizontal plane. In this embodiment, the axis of the blower 4's intake port 41 is parallel to the horizontal plane and both the first chamber 311 and the second chamber 312 are arranged on the same horizontal plane, with the airflow path being essentially planar (with no significant vertical flow), and from a top-down perspective, the airflow path within the noise-reducing air passage device 1 does not have any intersections.

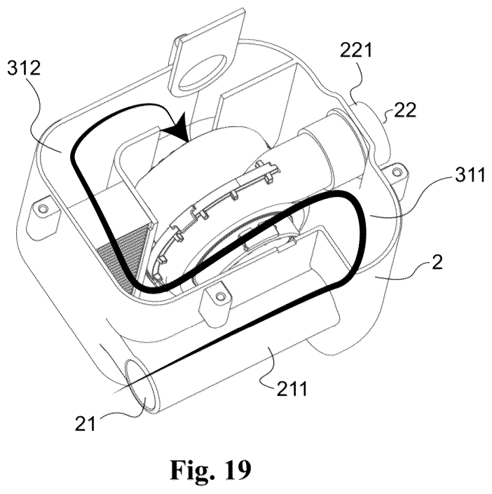

In another implementation, derived from Embodiment 2, a structure of the noise-reducing air passage device 1 is formed where the axis of the gas inlet 21 is parallel to the axis of the blower 4's intake port 41 (as shown in FIG. 19).

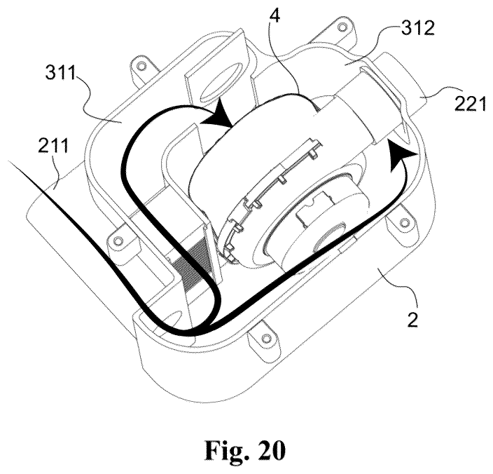

In another embodiment, derived from Embodiment 2, another structure of noise-reducing air passage device 1 is formed where the axis of the gas inlet 21 is perpendicular to the axis of the blower's intake port 41 (as shown in FIG. 20).

### Embodiment 3

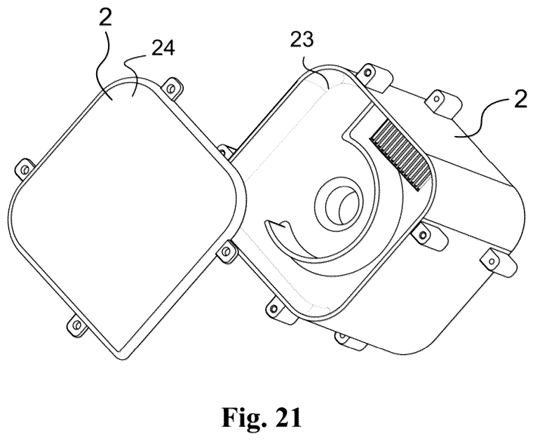

This embodiment provides a noise-reducing air passage device 1 used in ventilator systems, as shown in FIG. 21. This embodiment builds upon the noise-reducing air passage device 1 described in Embodiment 1, further specifying that the noise-reducing air passage device 1 has a wall near the intake port 41 of the blower 4 that is essentially coaxial with the intake port 41 of the blower 4 to guide the airflow (including circular and elliptical shapes). Specifically, this wall is located in a second chamber 312, which does not house the blower 4. The wall can be connected either to the wall near the blower 4's intake port 41 in the second chamber 312 (i.e., the wall separating the first chamber 311 from the second chamber 312) or to the wall of the housing 2 away from the blower 4's intake port 41 (i.e., the inner wall of the housing 2 in contact with external air). It is integrally formed with the housing. This wall allows the airflow to follow a curved path into the blower 4's intake port 41. As the breathable gas flows into the chamber 31 through the gas passage 3, it moves along the wall, aligning its flow path with the rotational direction of the blower 4's impeller before entering the blower 4. This design ensures that the airflow before entering the blower 4 maintains continuity with the flow path inside the gas passage device 3 of the noise-reducing air passage device 1, enhancing gas flow stability and reducing turbulence and noise, thereby further lowering the noise level of the noise-reducing air passage device 1.

In another implementation, the wall approximately coaxial with the blower 4's intake port 41 within the gas passage 3 of the noise-reducing air passage device 1 is an external baffle structure protruding into the chamber 31. This baffle structure is not integrally formed with the housing 2. The connection between the baffle and the housing 2 can be a physical connection, such as clips or hooks, or a chemical connection, such as glue or tape.

### Embodiment 4

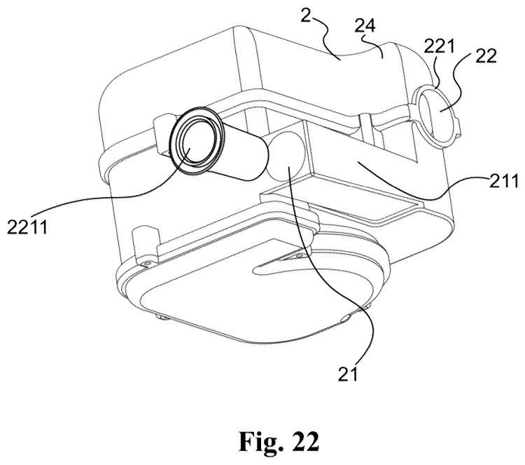

This embodiment provides a noise-reducing air passage device 1 for use in ventilators, as shown in FIG. 22. This embodiment includes a three-dimensional schematic diagram of the noise-reducing air passage device 1. In the embodiment shown in FIG. 22, building on the structure of the noise-reducing air passage device 1 from Embodiment 1, a trumpet-shaped elastomer 2211 is installed at the gas inlet 21. The tapered, from wide to narrow, channel structure has been tested and proven effective, and thus this structure is separated and configured in a trumpet shape at the gas inlet 21. Elastic materials can absorb energy from vibrations and sound waves, reducing noise caused by resonance, making this structure with elastic material more effective in noise reduction than rigid materials. Installing this structure made of elastic material at the gas inlet 21 results in a secondary noise reduction, reducing noise by at least 0.5 decibels compared to the structure described in Embodiment 1. The gas inlet 21 has a trumpet-shaped elastomer 2211, configured to smoothly (without causing turbulence or vortices) guide the breathable gas into the chamber 31. Specifically, the trumpet-shaped elastomer 2211 diffuses and disperses the airflow at the gas inlet 21, adjusting the flow rate to a more suitable level, thereby reducing turbulence and velocity differences in the airflow, making the gas entry into the chamber 31 smoother and more stable. This smooth flow of air effectively reduces impacts and vibrations within the chamber 31 and can also offset the sound waves at the gas outlet 22 to some extent, thus lowering noise production.

In another implementation, the trumpet-shaped component at the gas inlet 21 of the noise-reducing air passage device 1 is made of a non-elastic material and is integrally molded with housing 2.

In another implementation, at least part of the inlet pipe 211 of the noise-reducing air passage device 1 is made of an elastic material.

### Embodiment 5

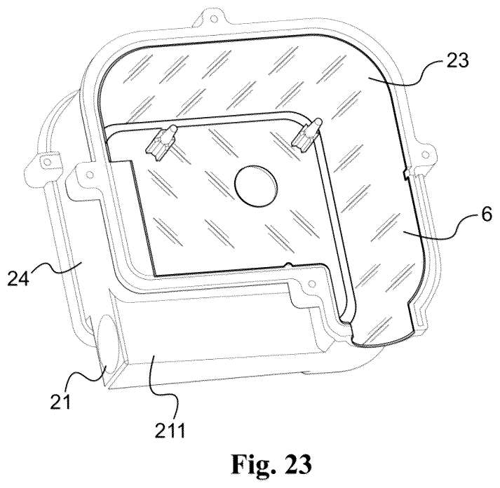

This embodiment provides a noise-reducing air passage device 1 for use in ventilators, as shown in FIG. 23. This embodiment includes a three-dimensional schematic diagram of the noise-reducing air passage device 1. In the embodiment shown in FIG. 23, the difference from Embodiment 1 is that the gas passage 3 of the noise-reducing air passage device 1 includes Polymer foaming materials. Although this disclosure has already demonstrated that the noise-reducing air passage device 1 can achieve regulatory noise levels even without Polymer foaming materials in the gas passage 3, individual needs and preferences of different patients may vary. Some patients may require or prefer a quieter environment for their respiratory machines. Therefore, while ensuring patient safety, and considering individual needs and preferences, soundproofing material 6 can be used inside the gas passage 3 of the noise-reducing air passage device 1 to further reduce noise levels. It should be noted that the soundproofing material 6 mentioned here does not include foam but consists of safer materials like silicone and gel. Silicone, gel, and other materials also possess noise-reducing and vibration-damping characteristics, capable of effectively absorbing and reducing noise in the airflow, thus providing a quieter and more comfortable breathing environment for patients. This selective use of soundproofing material 6 allows for better meeting the needs of different patients. This flexible design approach offers a more personalized and considerate respiratory therapy experience, further enhancing the sleep quality and life quality of patients.

### Embodiment 6

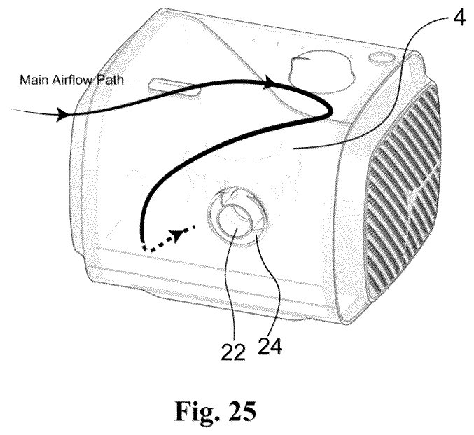

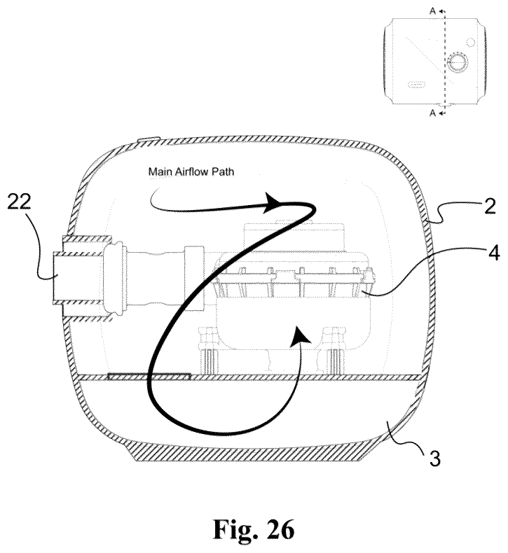

This embodiment provides a noise-reducing air passage device 1 for use in ventilators. See FIGS. 25 and 26. This embodiment includes a three-dimensional schematic diagram of the respiratory machine. In the embodiment shown in FIGS. 25 and 26, the difference of the noise-reducing air passage device 1 from Embodiment One is that the housing 2 of noise-reducing air passage device 1 forms part of the respiratory machine. This can also be understood to mean that in this embodiment, the blower 4 is at least partially exposed within the internal outer casing of the respiratory machine, such that the housing 2 of the noise-reducing air passage device 1 is at least partially the same as the outer casing of the respiratory machine (ventilator). The term “at least partially” can specifically be understood to mean that the housing 2 of the noise-reducing air passage device 1 could be entirely the same as the outer casing of the respiratory machine, or it could be that half or more or less of the housing 2 of the noise-reducing air passage device 1 is the same as the outer casing of the respiratory machine. FIGS. 25 and 26 illustrate schematic diagrams where the housing 2 of the noise-reducing air passage device 1 is entirely the same as the outer casing of the respiratory machine. This configuration of the respiratory machine, while meeting noise reduction requirements, reduces the machine's size. Smaller machines facilitate certain specific use scenarios, such as situations where some patients may need to frequently carry the machine. The smaller size of the machine ensures that these patients can continue treatment for respiratory-related disorders without interruption in special circumstances, aiding in the compliance with the machine treatment. This approach also simplifies the machine's structure by reducing connections between the machine's outer casing and the housing 2 of the noise-reducing air passage device 1, making it a more advantageous implementation in terms of manufacturing and subsequent maintenance.

Implementing the noise-reducing air passage device of this disclosure discussed herein provides at least the following benefits:

1) The design of a foam-free noise-reducing air passage device enhances the safety of respiratory machines (such as ventilators). In 2021, a globally renowned brand issued its first worldwide recall notice involving some of its Bi-level Positive Airway Pressure (BiPAP) devices, Continuous Positive Airway Pressure (CPAP) devices, and mechanical ventilators, followed by several more recalls. The recalls were primarily due to the use of sound-dampening foam in the noise-reducing air passage device, which could release particles and volatile organic compounds. In 2023, the FDA received thousands of complaints about this internationally known brand's CPAP and BiPAP machines. This incident had a significant negative impact on the brand's reputation.

The FDA requires that ventilators must demonstrate a noise level below 30 dB for market approval. Using foam for noise reduction is currently the simplest method because foam materials are easily obtainable and manufacturable. Their unique porous structure and material properties can convert noise into minimal energy, thereby reducing noise. Indeed, using foam to reduce noise can achieve a good noise reduction effect, and placing foam inside the noise-reducing air passage device is the simplest, most effective, and common means to meet regulatory noise standards.

Therefore, nearly all respiratory machines on the current market incorporate foam within the gas passage for noise reduction. However, foam can easily cause health issues for several reasons:

A. Due to the softness and relatively loose surface of foam materials, they can be easily worn away or peeled off by airflow during use, releasing particles. Once released, these particles can easily enter the patient's airway with the airflow, irritating the respiratory system. This may cause respiratory problems, leading to symptoms such as sore throat and coughing, especially in individuals already suffering from respiratory diseases such as asthma or Chronic Obstructive Pulmonary Disease (COPD).

B. Additionally, foam is often made from synthetic materials that may contain residual chemical additives. These chemicals can gradually be released as the foam ages and degrades. In some cases, if foam particles carry harmful microbes, they could lead to potential infections, particularly in individuals with weakened immune systems. Foam particles may also trigger allergic reactions, including sneezing, flu-like symptoms, and eye irritation.

C. Furthermore, foam used over long periods can accumulate dust, bacteria, and other contaminants, especially in respiratory machines. The device can easily inhale contaminants from the air, leading to bacterial growth and increasing the risk of infection.

This disclosure discussed herein specifically focuses on the safety and reliability of the ventilator's noise-reducing air passage device during design, implementing a series of stringent safety measures, including a foam-free design of the noise-reducing air passage device to reduce potential health risks to patients using ventilators. In designing the gas passage and filtration system, foam-free or easily replaceable foam designs are used to mitigate these potential health risks. For patient health and safety, the gas passage is designed to be foam-free. Since there is no foam in the gas passage, it reduces the chance of accumulating minute foreign objects, helping to maintain cleanliness in the gas passage. More importantly, the air breathed is not affected by minute residues from the foam itself, lowering the number of particles that patients might inhale or come into contact with, ensuring safety during device use. This is particularly important for patients who use the device over a long period as it helps to reduce potential respiratory issues. Additionally, some patients may be allergic to particles from materials such as foam, and the foam-free design reduces the risk associated with allergic reactions. This is also crucial for those allergic to foam materials or sensitive to chemically treated materials. With these characteristics, this product has enhanced patient safety and comfort during device use.

2) The design of the foam-free noise-reducing air passage device enhances reliability and extends the lifespan of the device. Foam is typically made from synthetic materials like polyurethane and polyether, which are softer compared to plastics and silicone and more susceptible to mechanical damage and chemical erosion. Foam is highly prone to aging due to environmental factors and often contains chemical additives or components that can alter its performance. These characteristics result in a shorter lifespan for foam materials, theoretically necessitating frequent replacements to maintain their effectiveness in noise reduction within the air passage device. In contrast, materials such as silicone and plastic usually have better wear resistance, corrosion resistance, and durability. They are less likely to be affected by environmental conditions and generally have better chemical stability, making them less susceptible to chemical factors and thus longer-lasting. The foam-free design of the noise-reducing air passage device eliminates foam, thereby extending the lifespan of the noise-reducing air passage device and, consequently, the device itself. Additionally, the foam-free design simplifies the internal structure of the device's gas passage by eliminating the need for extra components to secure foam, reducing the number of components and thus lowering manufacturing complexity. This helps enhance the device's reliability and stability. Moreover, foam materials often require timely replacement and cleaning to ensure health, but foam placed inside devices cannot usually be replaced or cleaned. The foam-free design avoids these steps, reducing the maintenance needs within the gas passage and increasing the convenience of using the device.

3) By employing effective noise-reducing structures, the noise-reducing air passage can achieve regulatory noise levels even without foam assistance. This noise-reducing air passage device utilizes various structures that effectively reduce noise, replacing the traditional role of foam within the air passage device. Specifically, the disclosure discussed herein employs multiple noise-reducing structures including, but not limited to, a conical inlet pipes, a trumpet-shaped elastomer made from flexible materials, a more reasonably placed blowers, an arc-shaped wall that aligns the airflow into the blower's intake port and is essentially coaxial with it, and curved corners on the inner wall of the gas passage that are aligned with the airflow path. The use of these structures and components not only effectively lowers noise levels but also enhances the stability and reliability of the noise-reducing air passage device. More importantly, the noise-reducing components within the air passage device, the internal structure of the air passage device, and the relationships among various structures are all based on substantial experimental data support and scientific analysis, ensuring the scientific validity and credibility of the noise-reducing air passage design. This scientifically credible noise-reducing air passage device design not only enhances the product's performance but also provides patients with a quieter and more reliable user experience.

4) The disclosure discussed herein reduces the cost of the device and aligns with environmental principles by using simpler materials and structures. The design of the noise-reducing air passage device in this disclosure discards traditional foam materials, utilizing only plastic components for the housing of the noise-reducing air passage device and as materials for forming chambers and passages, and silicone materials for a fixed connection between the blower and the housing of the noise-reducing air passage device, thus reducing the cost for producers to purchase additional foam materials. Compared to the more complex foam-containing structures in ventilators' noise-reducing air passage devices available on the market, this design, comprised of only two materials, is easier to process and assemble, making its manufacturing process simpler and more efficient. Additionally, by reducing the use of auxiliary materials like foam, it also lessens the environmental impact, aligning with modern society's environmental requirements and trends and making a positive contribution to environmental protection. Since foam releases harmful chemicals during production and disposal, which pollute the environment, the foam-free design of the noise-reducing air passage device avoids the release of these harmful substances, reducing negative environmental impacts. Therefore, this foam-free design using only silicone and plastic not only reduces the cost of the device but also adheres to ecological principles, representing a more sustainable and economical design solution.

The embodiments of the disclosure have been described in conjunction with the accompanying drawings, but the disclosure is not limited to the specific embodiments described above. The specific embodiments are merely illustrative and not restrictive. It must be noted that as used herein and in the appended claims, the regular forms “a” “an” “the” include their plural equivalents, unless the context clearly dictates otherwise. Those of ordinary skill in the art, inspired by this disclosure, can make numerous modifications without departing from the scope and spirit of the disclosure as protected by the claims, all of which fall within the protection of the disclosure.

1. A noise-reducing air passage device for use in a respiratory machine having a horizontal plane and a vertical direction, configured to pressurize gas and provide the pressurized gas to a patient's airway, the noise-reducing air passage device comprising:
a blower including an intake port and an exhaust port, configured to generate a flow of the pressurized gas;
a housing including a gas inlet configured to receive breathable gas, a gas outlet configured to allow the pressurized gas to flow out, an inner wall, and an outer wall, wherein the gas inlet and the gas outlet are at different heights; and
a gas passage, a space surrounded by the inner wall of the housing, configured to allow the breathable gas to flow through, wherein the gas passage forms at least two chambers within the inner wall, including a first chamber and a second chamber, the blower being located in the first chamber, and a total volume of the first chamber and the second chamber is more than three times a volume of the blower,
wherein an outlet pipe is provided at the gas outlet, and the outlet pipe is configured to connect to the gas outlet and communicate with the exhaust port of the blower,
a distance between the intake port of the blower and the opposing inner wall of the housing is greater than 5 mm,
the first chamber and the second chamber overlap in the vertical direction,
an axis of the intake port of the blower is perpendicular to the horizontal plane, and
the gas passage does not include foam.

2-3. (canceled)

4. The noise-reducing air passage device according to claim 1, wherein an axis of the gas inlet is parallel or perpendicular to the axis of the intake port of the blower.

5. The noise-reducing air passage device according to claim 1, wherein an inlet pipe is provided at the gas inlet, and the inlet pipe is configured to connect to the housing and includes a taper.

6. The noise-reducing air passage device according to claim 1, wherein the housing forms a part of the respiratory machine.

7. The noise-reducing air passage device according to claim 1, wherein the housing of the noise-reducing air passage device includes one of the following materials: polypropylene, polycarbonate, polyethylene terephthalate glycol-modified-1,4-cyclohexanedimethanol ester, polyamide, or polyether ether ketone.

8. A noise-reducing air passage device for use in a respiratory machine having a horizontal plane and a vertical direction, configured to pressurize gas and provide the pressurized gas to a patient's airway, the noise-reducing air passage device comprising:
a blower including an intake port and an exhaust port, configured to generate a flow of the pressurized gas;
a housing including a gas inlet configured to receive breathable gas, a gas outlet configured to allow the pressurized gas to flow out, an inner wall, and an outer wall; and
a gas passage, a space surrounded by the inner wall of the housing, configured to allow the breathable gas to flow through, wherein the gas passage forms at least two chambers within the inner wall, including a first chamber and a second chamber the blower being located in the first chamber, and a total volume of the first chamber and the second chamber is more than three times a volume of the blower,
wherein an outlet pipe is provided at the gas outlet, and the outlet pipe is configured to connect to the gas outlet and communicate with the exhaust port of the blower,
the outlet pipe has at least one of the following characteristics:
a. the outlet pipe includes at least one section of a wall that is coaxial with an axis of the exhaust port of the blower; and
b. a length of the outlet pipe is equal to or greater than a diameter of the exhaust port of the blower,
a distance between the intake port of the blower and the opposing inner wall of the housing is greater than 5 mm,
the first chamber and the second chamber overlap in the vertical direction,
an axis of the intake port of the blower is perpendicular to the horizontal plane,
the gas passage does not include foam, and
an airflow path has at least one staggered section when viewed from a top view.

9. The noise-reducing air passage device according to claim 8, wherein the outlet pipe includes at least one section of the wall near the exhaust port of the blower that is coaxial with the axis of the exhaust port of the blower, and a length of the at least one section of the wall is at least 6 mm.

10. The noise-reducing air passage device according to claim 8, wherein an inlet pipe is provided at the gas inlet.

11. The noise-reducing air passage device according to claim 8, wherein distances from a center of the intake port of the blower to four sides of the inner wall of the housing are approximately equal.

12. The noise-reducing air passage device according to claim 8, wherein the housing has a section of the wall near the intake port of the blower that is substantially coaxial with the intake port of the blower.

13. The noise-reducing air passage device according to claim 8, wherein the outlet pipe includes one of the following materials: plastic, silicone, rubber, thermoplastic elastomer, thermoplastic polyurethane, or fluororubber.

14. The noise-reducing air passage device according to claim 8, wherein the housing forms a part of the respiratory machine.

15. A noise-reducing air passage device for use in a respiratory machine having a horizontal plane and a vertical direction, configured to pressurize gas and provide the pressurized gas to a patient's airway, the noise-reducing air passage device comprising:
a blower including an intake port and an exhaust port, configured to generate a flow of the pressurized gas;
a housing including a gas inlet configured to receive breathable gas, a gas outlet configured to allow the pressurized gas to flow out, an inner wall, and an outer wall; and
a gas passage, a space surrounded by the inner wall of the housing, configured to allow the breathable gas to flow through, wherein the gas passage forms at least two chambers within the inner wall, including a first chamber and a second chamber the blower being located in the first chamber, and a total volume of the first chamber and the second chamber is more than three times a volume of the blower,
wherein an inlet pipe is provided at the gas inlet, configured to include an intake end connectable to the housing and an outlet end to discharge the breathable gas, wherein the inlet pipe has at least one of the following characteristics:
a. a straight-line distance from a center of the outlet end of the inlet pipe to a center of the intake port of the blower being between 35 mm to 150 mm; and
b. a ratio of a length of the inlet pipe to the straight-line distance from the center of the outlet end of the inlet pipe to the center of the intake port of the blower being between 0.16 to 2.29,
a distance between the intake port of the blower and the opposing inner wall of the housing is greater than 5 mm,
the first chamber and the second chamber overlap in the vertical direction,
an axis of the intake port of the blower is perpendicular to the horizontal plane,
the gas passage does not include foam, and
an airflow path has at least one staggered section when viewed from a top view.

16. The noise-reducing air passage device according to claim 15, wherein the inlet pipe is provided at an edge portion of the gas passage.

17. The noise-reducing air passage device according to claim 15, wherein a transitional component is provided between the gas outlet and the exhaust port of the blower, and is configured to connect the two and prevent gas leakage.

18. The noise-reducing air passage device according to claim 15, further comprising a wall that isolates the first chamber from the second chamber, and wherein the wall includes an opening and is configured to communicate with the intake port of the blower.

19. The noise-reducing air passage device according to claim 15, wherein an axis of the gas outlet is not located on a horizontal plane of an axis of the inlet pipe.

20. The noise-reducing air passage device according to claim 15, wherein the noise-reducing air passage device does not contain any components made from polymer foaming materials.

21. The noise-reducing air passage device according to claim 15, wherein the housing forms a part of the respiratory machine.

22. A noise-reducing air passage device for use in a respiratory machine having a horizontal plane and a vertical direction, configured to pressurize gas and provide the pressurized gas to a patient's airway, the noise-reducing air passage device comprising:
a blower including an intake port and an exhaust port, configured to generate a flow of the pressurized gas;
a housing including a gas inlet configured to receive breathable gas, a gas outlet configured to allow the pressurized gas to flow out, an inner wall, and an outer wall, wherein the gas inlet and the gas outlet are at different heights; and
a gas passage, a space surrounded by the inner wall of the housing, configured to allow the breathable gas to flow through, wherein the gas passage forms at least two chambers within the inner wall, including a first chamber and a second chamber the blower being located in the first chamber,
wherein an inlet pipe is provided at the gas inlet, configured to include an intake end connectable to the housing and an outlet end to discharge the breathable gas, and an area enclosed by a wall around the intake end of the inlet pipe is not less than 75% of an area of the intake port of the blower,
gas inlet and the gas outlet are not on a same wall,
a distance between the intake port of the blower and the opposing inner wall of the housing is greater than 5 mm,
the first chamber and the second chamber overlap in the vertical direction,
an axis of the intake port of the blower is perpendicular to the horizontal plane, and
the gas passage does not include foam.

23. The noise-reducing air passage device according to claim 22, wherein the intake end of the inlet pipe includes a trumpet-shaped elastomer, configured to smoothly guide the breathable gas into the at least one chamber.

24. (canceled)

25. The noise-reducing air passage device according to claim 22, wherein a distance between the outlet end of the inlet pipe and the opposing inner wall of the housing is greater than 1.5 times a diameter of the intake end of the inlet pipe.

26. The noise-reducing air passage device according to claim 22, wherein the inlet pipe has a draft angle, and the draft angle is at least 1.5°.

27. The noise-reducing air passage device according to claim 22, wherein the gas inlet and the gas outlet are non-coaxial.

28. The noise-reducing air passage device according to claim 22, wherein the housing forms a part of the respiratory machine.
